(19) United States
(12) Patent Application Publication
(10) Pub. No.: US 2006/0110481 A1
(43) Pub. Date: May 25, 2006

(54) COMPOSITIONS CONTAINING VITEX AGNUS-CASTUS BERRY OIL FOR COSMECEUTICAL APPLICATIONS AND METHOD OF ISOLATION OF THE ACTIVE COMPONENT

(75) Inventors: Muhammed Majeed, Piscataway, NJ (US); Farha Shaheen, Bangalore (IN); Subbalakshmi Prakash, Piscataway, NJ (US)

Correspondence Address: SABINSA CORPORATION, 70 ETHEL ROAD WEST, UNIT 6, PISCATAWAY, NJ, 08854, US

(73) Assignee: SAMI LABS LIMITED, Bangalore (IN)

(21) Appl. No.: 10/904,659

(22) Filed: Nov. 22, 2004

(51) Int. Cl. A61K 36/87

(52) U.S. Cl. 424/777

(57) ABSTRACT

The invention relates to the effective use of compositions containing a specially prepared oil from Vitex agnus castus berry as a potent anti-acne agent which is to be applied topically. The invention also describes the process of fractionation of the oil, identification of the active component using bioautographic technique, and the use of the active component as an anti-acne agent.

### FIELD OF INVENTION

The current invention discloses the new and unexpected finding that Vitex agnus castus berry oil is an effective antibacterial agent against Propionibacterium acnes, an anaerobic bacterium which is associated with Acne vulgaris. The invention describes the antibacterial activity of the Vitex berry oil and also that of its active component. The topical application of Vitex agnus-castus berry oil compositions in the treatment of acne vulgaris is described. The invention also describes the bioautographic technique used to test the antibacterial activity of the separated active fraction of the oil and also its major active component. Further the invention also describes a process to obtain the active component from Vitex berry oil.

### BACKGROUND OF INVENTION

Vitex agnus castus berry/fruit also known as chaste berry, Monk's pepper, wild pepper, Indian spice, renuka beej is indigenous to the Mediterranean, Central Asia, Greece and Italy. This plant has been used in traditional medicine for centuries. The medicinal uses of chaste tree are well documented in Greek and Roman texts. Both Hippocrates and later Dioscorides in his De Materica Medica have written in AD 77 about using Vitex as a sitz bath for diseases of the uterus. Throughout the Middle Ages in Europe Vitex was used to treat female hormonal disorders.

In Egypt and Nigeria it is used to treat hysteria and depression. In Unani medicine, this plant is used as a contraceptive and emmenagogue. Various medicinal properties of this plant are described in Indian systems of medicine, Ayurveda and Siddha. It is good for treatment of inflammation, catarrh, headache, stomach ache and is abortificient (Nadkarni, 1976).

The major application of Vitex agnus castus is in the management of premenstrual syndrome (PMS) in women (Schellenberg, 2001).

### DESCRIPTION OF PRIOR ART

DE 19844836 reveals a pharmaceutical composition of Vitex agnus castus, Cimicifuga racemosa, Zingiber officinale for the treatment of migraine.

AU 3433802 describes the use of Vitex extract to treat conditions of PMS, coronary heart disease (CHD) and osteoporosis particularly affecting women.

U.S. Patent application 20030026851 describes the composition for improvement of fertility health in female and male animals and humans. The invention is directed to supplemental combination of chaste berry along with Green Tea, vitamin E, selenium, L-arginine, folic acid, vitamin B6, B12, iron, zinc and magnesium for women. Vitex agnus castus enhances hormonal balance by increasing progesterone release and therefore ovulation frequency. The antioxidant Green tea, vitamin E and selenium improve overall reproductive health. L-arginine, an amino acid, stimulates the reproductive organs by improving circulation. Folic acid, vitamin B6 and B12, iron, zinc and magnesium help promote women's fertility.

DE 1 01 27897 describes a pharmaceutical formulation for treatment of osteoporosis or related disorders, comprising core of dried plant extract enclosed in sheath of calcium salt.

U.S. Patent application 20020068100 discloses a composition comprising ingredients of n-6 fatty acid, n-3 fatty acid, Vitamin E and extract of Viburnum opulus bark, extract of Vitex agnus castus berry and bioflavonoids for PMS symptoms and for treating irritable bowel syndrome or interstitial cystitis.

RU 21 44370 reveals development of homeopathic drug that can be used for treatment of patients with prostrate hypertrophy. Composition consists of Serenoa serulata, Pulsatilla pratensis, Thuja occidnetalis, Clematis recta, Vitex agnus castus taken at equal ratios and at dilution C 12, respectively, Composition is homeopathic drug as sugar granules. Homeopathic drug improves the general state of patients, shows good tolerance difficulty in urination.

DE 4305452 describes the use of extract of the plant Vitex agnus castus for stimulating dopamine receptors in central ganglia and thus for the treatment of Parkinson's syndrome.

U.S. Pat. No. 6,242,012 reveals the herbal composition which can be used to alleviate the symptoms associated with hormonal balance and normal bone growth in women contains supercritical extracts of Ginger, Rosemary, Black cohosh root, Chaste berry and Dong qui root extract and Schizandra berry.

The use of orally administered Vitex agnus-castus extract in the management of acne is reported in literature (Amann W., (1975); Amann, W., 1967). However, prior art does not report the topical use of Vitex agnus-castus oil in the management of acne.

### REFERENCES

A. K. Nadkarni. Indian Materia Medica (Volume 1) 1278-1279 1976.
Schellenberg R. Treatment for premenstrual syndrome with agnus-castus fruit extract: prospective, randomized, placebo controlled study. British Medical Journal 2001; 322: 134-137
Leyden J. J. ‘New understandings of the pathogenesis of acne’. J. Am. Acad, Dermatol 1995, 32: S15-S25.
Horvath, G, Kocsis, B, Botz, L, Nemeth, J, Szabo, L. “Antibacterial activity of Thymus phenols by direct bioautography” Acta Biologica Szegediensis. 2002, 46(3-4): 145-146.
Nagle D G, Wedge D E. 2000. “A new 2 D—TLC Bioautography method for the discovery of novel fungicidal agents to control plant pathogens”. Tektran; United States Department of Agriculture, Agricultural Research Service.
Homans, A L and Fuchs, A. “Direct bioautography on thin layer chromatograms as a method for detecting fungitoxic substances”. Journal of chromatography. 1970, 51:325-327.
Al Hadithi H T and Khudaier B Y. ‘Enzyme Production and antibiotic susceptibility of Propionibacterium acnes and P. granulosum from acne vulgaris patients and healthy persons’. Qatar University Science Journal 1996, 16 (2): 275-278.
Credito K L., Ednie L M., Jacobs M R and Appelbaum P C, 1999. ‘Activity of telithromycin against anaerobic bacteria compared to those of eight other agents by time kill methodology’. Antimicrobial Agents and Chemotherapy 43 (8); 2027-2031.
Fluhr J W, Gloor M., Dietz P. and Hoeffler U. 1999. ‘In vitro activity of 6 antimicrobials against Propionibacterium isolates from untreated acne papulopustulosa. Zentralblatt fuer Bakteriologie 289 (1): 53-61.
Lund, B M and Lyon G D. 1975. ‘Detection of inhibitors of Erwinia Carotovora and E. herbicola on thin layer chromatograms”. Journal of Chromatography. 110; 193-196.
Amann, W. Improvement of acne vulgaris following therapy with agnus castus (Agnolyt) Ther Ggw. 1967 January; 106(1):124-6.

### SUMMARY OF THE INVENTION

It is the objective of this invention to develop a potent natural product from Vitex agnus castus berry for the treatment of acne vulgaris, by topical application. It is another object of the present invention to disclose the use of the active component of the Vitex berry oil as an antibacterial agent against P. acnes.

It is still another object of the present invention to develop a process for obtaining active fraction of the Vitex berry oil and also to develop the bioautographic technique to elucidate the antibacterial activity against the anaerobic bacterium P. acnes.

Furthermore, it is the object of the present invention to analyze the active fraction of Vitex berry oil by NMR spectroscopy (Given in Annexure—1).

### BRIEF DESCRIPTION OF THE DRAWINGS

FIG. 1 is a photograph showing the chromatogram of Artemetin

FIG. 2 is a photograph showing the bioautography of Artemetin

### DETAILED DESCRIPTION

Acne is a chronic inflammatory disease of the pilosebaceous gland characterized by papules, pustules and cysts. It is common phenomenon in adolescent humans. It leads to psychological problems in patient, decreased social activity and development of psychological diseases. The causes for acne are sebaceous glands, androgens, keratin plugging/hypercornification and the major causative agent of acne vulgaris is an anaerobic bacterium called Propionibacterium acnes, which thrives in the microhabitat of sebaceous follicles. There are several synthetic topical drugs available for acne. However, there is major need to find out herbal antiacne compound. Vitex agnus castus berry is having effective role to enhance hormonal balance in women. There are few reports regarding the treatment of acne vulgaris by systemic therapy by this herb. But there are no reports on the topical treatment of acne vulgaris by this product. In order to find out active component in Vitex berry various extract and fractions of different polarities were evaluated through antibacterial studies and bioautographic technique.

Here we have carried out the in vitro studies on the antibacterial activity of Vitex oil, extracted from the fruits, against Propionibacterium acnes which is the major causative agent of acne vulgaris. Propionibacterium acnes is an anaerobic, pleomorphic rod shaped bacterium found in the clinical specimens. In the human body, P. acnes thrives on areas most exposed to air, such as the face and the nose. Its ability to live as an anaerobic in an air exposed environment comes from the fact that P. acnes lives in the microhabitat of sebaceous follicles thus causing acne vulgaris (Leyden, 1995). It grows typically as an obligate anaerobe.

Acne can be suppressed with topical and oral antibiotics. But most of the strains of P. acnes have developed resistance to the antibiotics that are commonly used today. So, new natural products are in major need for the cure of acne vulgaris. (Al Hadithi et al. (1996); Credito et al. (1999); Fluhr et al. (1999))

In order to find out the active component in the Vitex oil, the various components were fractionated and these fractions were subjected to direct bioautography technique.

Exemplary embodiments of the invention are presented.

### EXAMPLE 1

### Extraction of Vitex Berry Oil

Vitex berry powder is first extracted with non polar solvents selected from the list of solvents such as hexane, heptane, pentane, pet ether, benzene, toluene etc at 25 deg-70 deg C. Marc was removed by filtration through a nutsche filter. The filtrate was collected and distilled to get Vitex oil. The Marc was treated with aqueous methanol solvent with Hexane or EDC at 25 deg C.-70 deg C. which is called Vitex oil. Marc was treated with aqueous methanol solvent at 10-80 deg C. which is called Vitex berry extract. Both the fractions were subjected to testing for antimicrobial activity. The aqueous methanolic fraction of Vitex berry did not show any significant inhibitory activity.

### EXAMPLE 2

### Efficacy of the composition against Propionibacterium acnes

Vitex oil in different concentrations prepared in dimethyl sulphoxide (DMSO) as the solvent was taken for the studies. 1% clindamycin gel was used for comparison.

Anaerobic Chamber:

The anaerobic chamber [Model 8301-230, 3 ft polymer] used in the study is purchased from COY LABORATORY USA, which is provided with an automatic Air lock purge system.

The equipment consists of a main chamber wherein all the operations and incubation of the culture are carried out and a transfer chamber which is meant for taking the materials in and out of the equipment. The main chamber is provided with a heated fan box in order to maintain the incubation temperature (37° C.) and to circulate the air inside the chamber. A catalyst stakpak is fixed to this fan box which consists of palladium catalyst, which is meant for converting oxygen to water molecules by reacting with hydrogen molecules.

The anaerobic condition in the chamber is maintained by initialization with N₂ gas and then by passing the mixture of gases of N2+H₂+CO₂ in the proportion 80+10+10. Two gas tank arrangements is made wherein only N₂ is connected to the transfer chamber and mixed gas to the main chamber.

Gassing manifold: The gassing manifold is the equipment used for the preparation of the media by removing oxygen.

Preparation of Anaerobic Media:

The anaerobic media were prepared by heating the media while passing the mixture of gases of N₂ and CO₂ in 4:1 proportion simultaneously. The media were added with a redox indicator, which is colourless in the presence of anaerobiasis and shows blue colour in its absence. It was dispensed to 30/20/10 ml vials and sealed with rubbers and aluminium cramps and sterilized by autoclaving at 121° C. for 20 minutes.

Antimicrobial Studies:

Preparation of the inoculum: The culture from the broth was inoculated to the fresh medium in vials [10%] and incubated for 48 hours at 37° C. The optical density (O D) of the culture at 625 nm was measured. It was maintained between 0.64 and 0.80 which contains approximately 12×10⁶ cells/ml [4.0 McFarland standard].

Procedure: Medium used for this study was reinforced clostridial Agar (RCA). The medium which was prepared and sterilized in 30 ml vials was poured into the plates inside the chamber and allowed to solidity. The culture was inoculated [0.3 ml/plate] into the plates and spread. After 30 minutes, antibacterial sterile discs [6 mm] were dispensed (2/plate), and 2.5, 5.0, 7.5 and 10 μl of the prepared samples and controls were dispensed onto the discs. The plates were incubated inside the anaerobic chamber at 37° C. for 48 hours duration.

Antimicrobial Studies Against E. coli and S. aureus.

Antimicrobial studies of Vitex oil against general pathogenic aerobic bacteria such as E. coli and S. aureus were also carried out. E. coli was grown in lactose broth and S. aureus in nutrient broth. The method used was the same as it was followed for P. acnes. Clindac A (Clindamycin phosphate gel 1% w/w) was used as positive control. The clearence zone formed around the discs were measured and expressed in mm.

Results:

As seen in Table 1, it is confirmed that Vitex oil effectively inhibits Propionibacterium acnes in concentrations above 0.3% and the activity is well comparable with the Clindamycin Gel.

TABLE # 1

| | Concentration of the sample | Zone of inhibition [mm] | |
|---|---|---|---|
| Sl. No. | [% w/v] | Vitex oil | Clindamycin |
| 1. | 5 | 16 | 15 |
| 2. | 3 | 12 | 10 |
| 3. | 2 | 9 | 9 |
| 4. | 1 | 9 | 8 |
| 5. | 0.5 | 8 | 0 |
| 6. | 0.4 | 8 | 0 |

The aerobic bacteria E. coli and S. aureus are also inhibited by the Vitex oil at the concentrations above and also at 0.1 and 0.5% respectively (Table-2).

TABLE # 2
Antimicrobial effects of Vitex Oil

| | | Zone of Inhibition | Zone of |
|---|---|---|---|
| Sl. | Concentration of Vitex Oil | (mm) | Inhibition (mm) |
| No. | (% w/v) | E. coli | S. aureus |
| 1 | 5 | 15 | 12 |
| 2 | 1 | 10 | 9 |
| 3 | 0.5 | 9 | 8 |
| 4 | 0.1 | 8 | 0 |

### EXAMPLE 3

### Isolation and Identification of Active Fractions of Vitex Oil

TABLE # 3
Chemical composition of Vitex oil

| S. No | Chemical constituent | Contents | Percentage |
|---|---|---|---|
| 1 | Volatile Oil | — | 0.5% |
| 2 | Essential Oils | i) α-pinene | 0.012% |
| | | ii) Limonene | Not detected |
| | | iii) Cineol | Not detected |
| | | iv) Linalool | Not detected |
| | | v) 4-Terpineol | Not detected |
| | | i) β-caryophyllene | Not detected |
| 3 | Total fatty acids of oil | i) Lauric acid | 0.18% |
| | | ii) Myristic acid | 0.01% |
| | | iii) Palmitic acid | 1.62% |
| | | iv) Stearic acid | 1.445% |
| | | v) Oleic acid | 3.25% |
| | | vi) Linoleic acid | 13.48% |
| | | vii) Linolelic acid | 1.37% |
| | | Total | 21.36% |
| 4 | Total fatty alcohols | i) Tetracosanol | 0.052% |
| | | ii) Hexacosanol | 0.21% |
| | | iii) Heptacosanol | 0.31% |
| | | iv) Octacosanol | 1.44% |
| | | v) Triacontanol | 1.50% |
| | | Total | 3.51% |
| 5 | Other compounds | p-hydroxybenzoic acid | 0.001% |

The antibacterial effects of aqueous, methanolic Vitex agnus castus fruit extract and various extract fractions of different polarities were evaluated by bioautography technique. Non polar oil showed activity against Propionibacterium acnes. Further fractionation of oil has been done by Silica gel chromatography. Silica gel was been loaded more than half quantity of oil and fraction was collected from mobile phase, hexane, chloroform, 50% chloroform, chloroform, 10% methanol, 50% Methanol and Methanol alone. Active fraction was identified by bioautography technique.

Vitex oil obtained by extraction of non polar solvent at temperature ranging from 25 deg C. to 120 deg C. It was distilled by fractional distillation. The fractional distillation was carried out at various temperatures 60 to 300 deg C. most preferably 60 to 100 deg C. The atmospheric pressure was maintained between 1 to 10 mm, preferably 3 to 6 mm and active fraction was determined by bioautography technique.

This technique has been used in some laboratories for testing the antimicrobial activity of various natural products against the aerobic bacterial and fungal cultures (Hovarth, et al. (2002); Nagle, et al. (2000); Homans et al. (1970)

Materials and Methods:

Direct bioautography is the technique used to localize antibacterial activity on a chromatogram. (Lund et al. 1975)

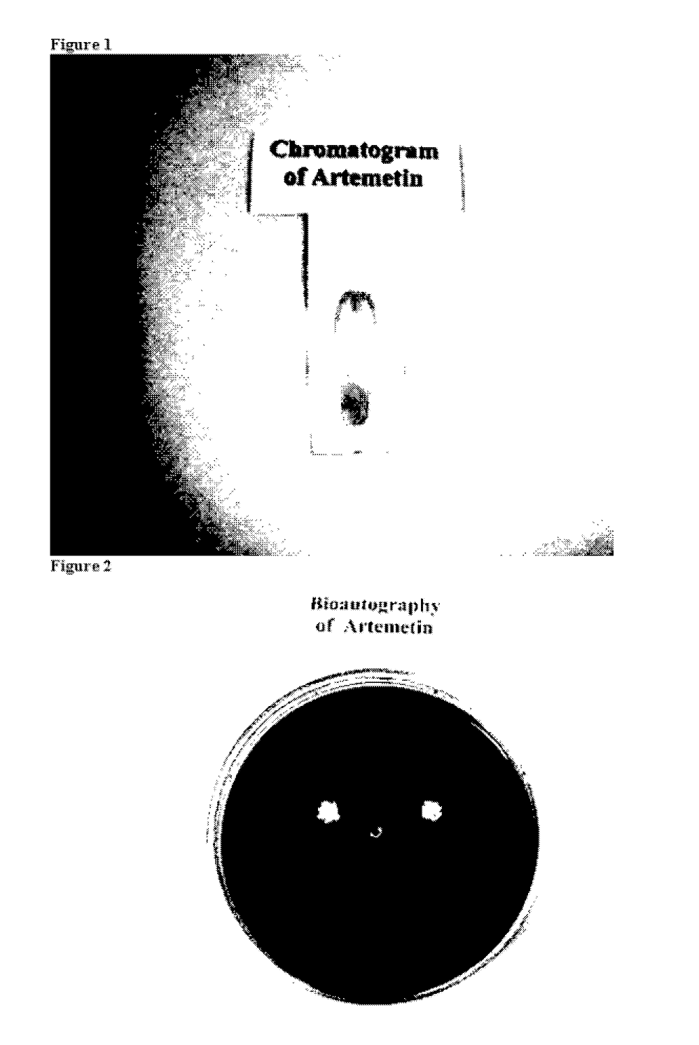

In the present study, the separated fractions of Vitex oil were subjected to Thin Layer Chromatography on silica gel. The solvent system used was hexane: chloroform in 7:3 proportion [FIG. 1]. These chromatograms were dried in the oven at 60° C. for 1 hour to eliminate the traces of solvents. This can be visualized either by spraying the thin layer chromatogram with 5% methanolic sulphuric acid followed by heat drying or under UV light. The plates were taken to the anaerobic chamber where the bioautography experiment was carried out. The medium used was reinforced clostridial agar medium. The culture used was Propionibacterium acnes ATCC 11827. O.D. of the inoculum at 625 nm was maintained between 0.64 to 0.8 [4.0 MC Farland standard] with the cell density of 1 2×10⁶/ml. The thin layer chromatograms were placed on the solidified agar medium in the petriplates with silica gel surface facing upwards. Onto this upper surface of the chromatograms, a thin layer of the molten agar medium inoculated with the organism was poured and the plates were incubated at 37° C. inside the chamber for 48 hours.

After incubation, 1-1.2 ml of tetrazolium salt solution [2% concentration] was sprayed as a coloring reagent on the plates. This reagent gave reddish purple colouration with the organism. The circular zones of clearance could be visualized clearly where there was no colouration. The diameter of the zones were measured and expressed in mm.

Nine fractions of Vitex oil were subjected to bioautography. Out of these, one fraction showed good inhibition of P. acnes with a clearance zone of 10 mm and another fraction showed moderate inhibition with a zone of 5 mm.

The inhibition zone in both cases appears at the basal part of the thin layer chromatogram. Other 7 fractions are not showing any inhibition.

The part of the chromatogram showing inhibition of 10 mm diameter appears to be consisting of about 3 compounds. From this active fraction, active compound was isolated by ethyl acetate:methanol crystallization process at 5-40 deg C. Yellow to orange needles/rectangular/prism crystals were obtained. Melting point ranges from 100 deg C. to 200 deg C. Crystals were identified and characterized by IR (Infrared Spectroscopy) and NMR (Nuclear Magnetic Resonance spectroscopy).

A compound was isolated from the active fraction with aqueous methanol at 30 to 35° C. It was crystallized and the structure and identification was done by IR & NMR.

Identification of Compound:

IR spectrum of compound shows hydroxyl (3500 cm⁻¹), conjugated carbonyl group (1666 cm⁻¹) C—H aromatic bend (67.18 cm⁻¹), C—O Stretch (1027.99 cm⁻¹), C—H stretch aromatic (3139.90 cm⁻¹, 3099.39 cm⁻¹, 3014.53 cm⁻¹, 3001.03 cm⁻¹) showed the structure is flavone. NMR spectrum showed presence of four aromatic proton and total 15 proton of 5-methoxy group.

The isolated single compound Artemetin which is in the form of crystals was tested for antibacterial activity by using bioautographic technique. Clearance zone was exhibited on the thin layer chromatogram (FIG. 2).

The concentration of Artemetin in Vitex oil has been determined by HPLC. The results obtained show that the concentration of Artemetin in Vitex oil is in the range of 0.1 to 0.5%.

The inhibitory activity of the isolated single compound Artemetin against E. coli and S. aureus have been also conducted. The studies show that the compound had MIC of 1.0% for both the organisms (Table-4).

TABLE # 4
Antimicrobial effects of Artemetin

| | | | Zone of |
|---|---|---|---|
| | Concentration of | Zone of Inhibition (mm) | Inhibition (mm) |
| Sl. No | Artemetin(% w/v) | E. coli | S. aureus |
| 1 | 5 | 12 | 10 |
| 2 | 1 | 9 | 8 |
| 3 | 0.5 | 0 | 0 |

What is claimed is:

1. A method of inhibiting microbial growth comprising contacting at least one microbe selected from the group consisting of Propionibacterium acnes, Staphylococcus aureus, Escherichia coli, and mixtures thereof with an oil composition isolated from Vitex agnus-castus fruit, wherein the oil composition comprises monoterpenes, fatty acids, fatty alcohols, p-hydroxybenzoic acid, artemetin, sitosterol.

2. The method of claim 1, wherein the oil composition comprises 0.01%-0.5% artemetin.

3. The method of claim 1 wherein the oil composition is isolated and purified by the steps of (a) extraction from Vitex agnus-castus fruit with low boiling solvent; (b) fractionation by column chromatography.

4. The method of claim 2, wherein the growth of Propionibacterium acnes is inhibited.

5. The method of claim 2, wherein the growth of Staphylococcus aureus is inhibited.

6. The method of claim 2, wherein the growth of Escherichia coli is inhibited.

7. A method of treating a patient having a microbial infection and in need of such a treatment, comprising administering an antimicrobial amount of an oil composition isolated from Vitex agnus-castus in the patient, wherein the oil composition comprises artemetin, monoterpenes, fatty acids, fatty alcohols, p-hydroxybenzoic acid, sitosterol.

8. The method of claim 7, wherein the oil composition is administered topically to at least a portion of the skin of the patient.

9. The method of claim 7 wherein the microbial skin infection treated is Acne vulgaris.

10. The method of claim 8 wherein the oil composition is used in concentrations of 0.4 to 25%.

11. The method of claim 1 wherein the composition is applied to the skin in the form of a lotion, cream, dilution, gel, emulsion, dispersion, transdermal patch, facial mask, or other topical formulation.
